(19) United States
(12) Patent Application Publication
(10) Pub. No.: US 2012/0191183 A1
(43) Pub. Date: Jul. 26, 2012

(54) MEDICAL VALVE IMPLANT, IN PARTICULAR HEART VALVE IMPLANT, FOR IMPLANTATION IN AN ANIMAL BODY AND/OR HUMAN BODY

(75) Inventors: Alexander Rzany, Nuernberg (DE); Matthias Fringes, Ansbach (DE); Alexander Borck, Aurachtal (DE); Robert Schmiedl, Hirschaid (DE); Claus Harder, Uttenreuth (DE)

(73) Assignee: BIOTRONIK AG, Buelach (CH)

(21) Appl. No.: 13/349,116

(22) Filed: Jan. 12, 2012

Related U.S. Application Data
(60) Provisional application No. 61/435,359, filed on Jan. 24, 2011.

(51) Int. Cl. A61F 2/24

(52) U.S. Cl. 623/2.17

(57) ABSTRACT

The invention relates to a medical valve implant (10a-d), in particular a heart valve implant (12a-d), for implantation in an animal body and/or human body, comprising a support frame (14a-d) which has at least one wall (16a-d). It is provided that one region (18a-d) of the at least one wall (16a-d) of the support frame (14a-d) is curable.

### CROSS REFERENCE TO RELATED APPLICATIONS

This invention claims benefit of priority to U.S. provisional patent application No. 61/435,359, filed Jan. 24, 2012; the contents of which are herein incorporated by reference in their entirety.

### TECHNICAL FIELD

The invention relates to a medical valve implant, in particular a heart valve implant, for implantation in an animal body and/or human body.

### BACKGROUND

Valve implants are used in medical applications for implantation in an animal body and/or human body permanently or at least for an extended period of time to perform replacement functions. Heart valve implants are known, for example, such as aortic valve implants that perform the function of the natural aortic valve. In that case, the valve implant is affixed after expansion of the implant structure immediately implantation, and assumes the position of the natural aortic valve.

A common problem is that the implant is affixed in an incorrect position, which can cause the implant to fail. This occurs frequently when calcification takes place, for example, i.e. the deposition of sodium salts, in particular calcium phosphate (hydroxyapatite) on the structures of the heart and, in particular, when an aortic stenosis has calcified in a highly asymmetrical manner.

### SUMMARY

The problem addressed by the invention is that of creating a medical valve implant that can be implanted exactly and reliably at an implantation site.

The invention relates to a medical valve implant, in particular a heart valve implant, for implantation in an animal body and/or human body, comprising a support frame that has at least one wall.

It is provided that at least one region of the at least one wall of the support frame is curable. By way of the embodiment according to the invention a valve implant can be provided that can be positioned in an optimal manner and anchored in a gentle manner. Furthermore, it can be advantageously adapted to the parameters or anatomical details of an implantation site, such as calcification of a blood vessel wall and/or an annulus, and/or another, hereditary and/or diseased anomaly of the implantation site. Moreover, it enables optimal placement at the site of the defective physiological valve. In addition, it is made possible to perform repositioning and permanent fixation in vivo. Furthermore, a pressure gradient of a flow medium acting on the medical valve implant, such as blood, can be kept homogeneous, which advantageously results in a minimal material load on the valve implant and, therefore, a minimal risk of fatigue since the valve opens in a uniform manner. In turn, this results in a long service life of the cusp and, therefore, the valve. Furthermore, better clinical results compared to conventional valve implants can be achieved by the improved functionality of the valve that can therefore withstand a higher pressure gradient in the presence of an asymmetrically calcified annulus. Due to the embodiment according to the invention, symmetry in the flow dynamics of the flow medium can be increased, which advantageously reduces the risk of further calcification. Furthermore, the valve implant is optimized in terms of flow mechanics, thereby making it possible to reduce turbulent flows of the flow medium, which, in turn, reduces the tendency for clots to form.

In this context, a “valve implant” is intended to mean a body in particular that functions as a replacement for a non-return valve, permanently or for an extended period of time after implantation. Any medical valve implant that appears suitable to a person skilled in the art, and that is implanted in a cavity, such as a digestive tract, a bronchial tract, and/or a blood vessel, such as an artery and/or vein in particular, would be feasible in this context. In this context, a “heart valve implant” is intended to mean, in particular, an implant structure, such as a pulmonary valve, a mitral valve, a tricuspid valve, and in particular an aortic valve made of natural and/or artificial material. Another implant structure that appears reasonable to a person skilled in the art would also be feasible, however.

Furthermore, a “support frame” in this context is intended to mean, in particular, a structure such as a cylinder or a hollow cylinder that substantially imparts a shape and/or form to the valve implant. The scope of protection should not be limited to a circular contour; square or oval shapes should also fall within the scope of protection. In addition, the hollow cylinder can also have openings in the jacket surface thereof. The support frame is preferably formed by a tubular polymer cylinder. It insulates the defective human valves against the new, usually animal-based valve material of the valve implant, thereby minimizing a contact surface between the artificial valve and an aortic wall in a manner that is gentle to tissue. Furthermore, calcification of the animal or artificial heart valve can be effectively prevented in this manner since the calcification could extend to the animal or artificial valves if they had direct contact with the defective human valves.

Furthermore, the valve implant is preferably passively expandable or can be expanded using a force applied from the outside, or is plastically deformable. The passive expansion can be accomplished in a structurally simple manner using a balloon catheter, wherein the support frame can be crimped onto a balloon catheter.

A “wall of the support frame” is intended to mean, in particular, an outer wall of the support frame, and preferably the jacket surface of the support frame which, in the implanted state, faces the direction of the implantation site or faces outwardly in the radial direction from the center point of the support frame. The purpose of the wall, in the implanted state, is to provide contact with the implantation site or an annulus, or it has contact with the tissue of the implantation site. Preferably the wall region extends at least in the circumferential direction around the entire circumference of the support frame. Furthermore, the wall region preferably extends along an entire axial length of the support frame.

In this context, “curable” is intended to mean, in particular, that a material of which the wall region is made transitions from a first, less viscous state into a second, more viscous, preferably solid state. The state transition can be dependent upon any physical, chemical, or electrical factor that appears reasonable to a person skilled in the art, such as time, temperature, radiation (IR, VIS, UV, gamma, radioactive radiation), ultrasound, magnetism, current, or a change in pH value, concentration, and/or charge. The material is advantageously more highly polymerized and/or crosslinked.

It is furthermore provided that the region of the at least one wall of the support frame can be cured using UV radiation. Preferably, a UV light source which can be controlled from the outside is positioned in the balloon catheter and can enable rapid curing of the polymer tube immediately after dilatation. The UV radiation is preferably in the wavelength range 240-400 nm, particularly preferably in the range 280-375 nm. By implementing UV-induced curing, a curing time of the support frame can be advantageously shortened compared to valve implants according to the prior art. Furthermore, this also makes it possible for the uncured state of the support frame to be maintained for a sufficiently long time period to ensure good adaptation of the valve implant to the morphology of the heart and/or a necessary repositioning.

Advantageously the at least one region of the at least one wall of the support frame is made of a curable material such as, but not limited to acrylates, methacrylates, cyanoacrylates, epoxides, urethanes, acrylamides, acyl acids. Preferably the support frame is formed entirely or 100% of a UV-curable, elastic polymer cylinder or tube. Advantageously, highly flexible acrylates and/or cyanoacrylates and methacrylates are used which cure in a time of 1-60 sec with application of 1-10 J/cm². In principle, any mixture of the materials that appears advantageous to a person skilled in the art would also be feasible. In addition, other materials such as casting resins, resins in general, polyethylene (PET), polycarbonate (PC), polyamide (PI), PEBAX®, PVP, PAll, silicone rubber, or PVC can also be added. Furthermore, it is also feasible to add other substances such as softening agents (e.g. triethanolamine), crystal nuclei (e.g. N-vinyl pyrrolidone), dyes (e.g. eosin Y), radical starters, and/or contrast medium. The embodiment according to the invention enables curing to take place using a simple design. Furthermore, a smaller implantation diameter can be achieved since the valve implant contains no metal or very little metal. Another result thereof is that the valve implant can be deformed in a highly flexible manner. Moreover, there is no direct contact of metals with the cardiac wall, thereby reducing interference with the propagation of electrical pulses on the heart compared to valve implants according to the prior art. In addition, various support frames that can be used in a flexible manner depending on the application can be provided by varying the materials.

Furthermore, it is advantageous if the support frame is provided to compensate for a difference in a shape of an inner cross section of the support frame and a cross-sectional area of an implantation site. “Provided” is intended to mean, in particular, specially equipped, designed, and/or prepared. In this context, a “shape of the inner cross section of the support frame” refers, in particular, to a largely round or cylindrical shape which enables the cusp of the valve to open and close without complication. A “cross-sectional area of the implantation site” in this context is intended to mean, in particular, a highly asymmetrical or non-circular site, in particular having a calcified aortic stenosis. The support frame thereby advantageously adapts the possible non-uniform shape of an outer diameter of the support frame to the cross-sectional area of the implantation site, thereby enabling the implant to account for the local details at the implantation site to particular extent. Advantageously as a result, an asymmetry of the blood vessel wall or the annulus can be compensated for, and a largely round, symmetrical inner shape of the support frame can be retained nevertheless to ensure the required flawless, complication-free function of cusps of the valve.

According to a further embodiment of the invention, the support frame comprises at least one means for mechanical reinforcement. These means or the support means can be a metallic (e.g. Nitinol) or polymeric structure which is integrated into the curable polymer, and/or it can be an embodiment such as a thickened region, a ridge, and/or an annular structure that is integrally formed onto the support frame and/or is formed therein. Basically the means for mechanical reinforcement can also be connected to the support frame in another way, such as a non-positive connection, a bonded connection, or a form-fit connection. The means for mechanical reinforcement is used in particular to support fixation of the valve implant, thereby making it advantageously possible to omit e.g. plug-like anchoring mechanisms according to the prior art, which irritate the tissue and can induce inflammation. Furthermore, the means for mechanical reinforcement can be used as positioning means to ensure exact positioning of the valve implant.

It is furthermore provided that the support frame comprises a stent. In this case the support frame preferably forms the stent which is therefore composed of a balloon-expandable, curable polymer. By designing the valve implant as a stent, or given that the support frame comprises a stent, a structure can be provided that is easily implanted in terms of the design thereof. This embodiment makes it possible to design a valve implant having a short installation height, thereby lowering the risk—compared to implants according to the prior art—e.g. of obstructing the flow of blood into the ostial passages of the coronary arteries, or even closing them entirely.

Furthermore, it would be advantageous if the valve implant comprised a further stent in addition to the support frame. This stent is disposed radially inwardly relative to the support frame, and can be any stent deemed usable by a person skilled in the art, e.g. a balloon-dilatable or self-expanding stent composed of an elastic or superelastic material, such as a metallic material (iron, magnesium, nickel, tungsten, titanium, zirconium, niobium, tantalum, zinc, silicon, lithium, potassium, calcium, manganese), a combination of a plurality of metallic materials, a memory-effect material such as Nitinol, of medical stainless steel such as Co-Cr-Stahl, of plastic, a ceramic, and/or a biodegradable material.

Furthermore, it can be advantageous for the means for mechanical reinforcement to be formed of at least one annular structure, thereby enabling the valve implant to be placed on the annulus and affixed thereto in a physiologically optimal manner using self-positioning. The annular structure can be formed entirely of the same curable material as the support frame and/or it can be formed by a hollow ring. In the latter case, a jacket of the ring is made of a flexible polymer material, preferably such as polyethylene (PET), polycarbonate (PC), polyimide (PI), PEBAX®, PVP, PAll, PVC and/or any other material that appears usable to a person skilled in the art.

Exact and secure fixation can be advantageously achieved when the annular structure is disposed on a proximal end and/or a distal end of the support frame. In this context, a “proximal end” is intended to mean in particular an end of the support frame that points toward the user during the implantation process. Therefore, a “distal end” is an end that points away from the user. Preferably an annular structure is disposed on the proximal end as well as on the distal end, thereby enabling the shape of the support frame to be adapted in a particularly reliable manner to the shape of the implantation site or the annulus.

It is also provided that the annular structure can be filled with a curable material. The curable material can be liquid in the filling state. In this case as well, the state transition from e.g. liquid and/or less viscous to viscous or solid can be dependent on any physical, chemical, or electrical factor appearing reasonable to a person skilled in the art, although preferably time and/or UV radiation. Advantageously a crosslinkable polymer solution is used. Preferably the polymer solution can be composed of a plurality of components, the relative ratio and composition of which can be advantageously selected to obtain specific physical properties. Optionally, different properties of the individual annular structures can also be obtained. Advantageously, the reaction speed can be influenced directly by the ratio of polymeric to monomeric portions and the concentration of chemically active substances (initiators of the polymerization).

Other properties can be achieved by adding fillers that do not participate in the polymerization, e.g. x-ray visibility can be achieved by adding contrast medium (e.g. barium sulfate), the flexibility of the polymerized filler can be increased by adding elastic polymers (e.g. rubber particles), the mechanical strength of the polymerized filler substance can be increased by adding fillers such as hydroxyapatite used in bone cements, and/or other properties deemed to be advantageous by a person skilled in the art. Optionally, for positioning, the annular structures can also be filled temporarily with physiological saline solution which is replaced by the curable polymer solution in a second step. A catheter system of the type described in WO 2006/105190 A2, which is characterized by a detachable connection, can be used to fill the annular structures. As an alternative, valves that can be controlled from the outside via the catheter system can be used; they establish a detachable connection between annular structures and catheter system.

Advantageously the medical valve implant comprises at least two annular structures which can be filled independently of each other, thereby enabling the valve implant to be positioned in a particularly precise manner. Furthermore, various properties of the annular structures or the filler substance can be achieved easily in this manner. For example, the annular structure on the distal end of the support frame is filled first using the catheter system connected to the implantable valve. It is intentionally inflated slowly to gradually position it in an optimal manner and therefore forms a stop for the optimal placement of the artificial heart valve that can be implanted in a minimally invasive manner. Once the distal annular structure has been placed correctly, the annular structure on the proximal end of the support frame is filled, and the heart valve is automatically centered in the region of the constriction formed by the defective physiological heart valve. The solution to be filled can be partially withdrawn via the catheter system to reduce pressure, thereby advantageously making it possible to reposition the annular structure(s) e.g. if they are mispositioned. Once the correct positioning has been verified, the valve implant can be permanently affixed by crosslinking the solution used for filling. Furthermore, a failure of the implant can be prevented in this manner using a simple design.

Moreover, the medical valve implant advantageously comprises a valve disposed above the annular structure in an axial direction on the proximal end of the support frame. Due to this design, a valve implant is provided in which the valve advantageously performs self-locating/self-positioning during implantation. Basically the valve could also be disposed axially between two annular structures. This results in a particularly reliable adjustment of the valve. When positioned between the annular structures, the valve can also be installed on the support frame and/or connected to an annular structure or to both annular structures. The valve can be connected to the annular structure or the support frame using any type of connection deemed to be reasonable to a person skilled in the art, such as suturing and/or bonding.

According to a preferred development, at least two annular structures in the intended end state are disposed axially upstream of and downstream of the annulus in the direction of flow of a flow medium. In this context, a “flow direction of a flow medium” refers, in particular, to the scientifically known flow direction of arterial and/or venous blood in the heart and, particularly advantageously in the case of the aortic valve, to the flow of blood from the left ventricle into the aorta. The annulus is preferably the aortic annulus. Due to the implementation of the embodiment according to the invention, the valve implant can be adapted particularly well to the anatomy of the heart or a heart valve region e.g. with an aortic bulb.

It is also provided that at least one communication structure is located between two annular structures. In this context, a “communication structure” is intended to mean in particular a structure having one or more hollow elements, such as a channel system and/or a cavity, using which the two annular structures have contact with each other. The communication system can be designed to be independent of the support frame or the cylinder, or can be integrated therein. In the latter case the cylinder is designed e.g. to have a double wall with a cavity therebetween. The filler for the annular structures and the communication structure have the same design. The connections between the individual functional units of the valve implant enable the crosslinkable polymer solution to be exchanged using a simple design, and thereby advantageously enables the valve implant to automatically adapt to the geometry of the heart. A largely automatic positioning of the valve in the annulus during filling with the curable polymer solution is therefore attained, as a substantial advantage.

According to a further embodiment of the invention, a support structure is disposed on at least one annular structure, on the proximal end of the support frame and/or on the proximal end of a valve. This support structure is preferably used to support, position, and provide proximal mechanical fixation of the valve. It is preferably designed as a metal framework in the form of a wire mesh, preferably of Nitinol, and/or as a further or third annular structure which can be positioned, filled, and affixed independently of the other functional units of the valve implant. By way of the support structure, the relative positioning of the annular structures and the valve cusps can be ensured, to guarantee that the cusps open and/or close exactly and without complication. The embodiment as a further annular structure is advantageous in particular when this annular structure has high mechanical flexibility and therefore enables good adaptation to the physiological details of the heart. As a result, optimal flow properties and low mechanical restriction of heart motion can be achieved.

Particularly advantageously, the medical valve implant is designed as an aortic valve, thereby making it possible to provide a refined replacement structure for the heart valve that malfunctions most often. Favorably, complications such as disruptions of the mitral valve or the need for a cardiac pacemaker can also be reduced. An embodiment as a pulmonary valve, tricuspid valve, or a mitral valve is likewise feasible.

Advantageously, a deposit-inhibiting, in particular calcification-inhibiting coating can be provided on the implant, in particular homocysteinic acid. The risk of a disruption or malfunction of the valve implant can therefore be reduced.

### DESCRIPTION OF THE DRAWINGS

The invention is explained in the following in greater detail as an example, with reference to an embodiment depicted in drawings. In the drawings:

FIG. 1 shows a valve implant according to the invention, in the expanded state,

FIG. 2 shows the valve implant in FIG. 1 installed on an implantation device,

FIG. 3 shows the valve implant in FIG. 1 when implanted at an implantation site,

FIG. 4 shows the valve implant in FIG. 1, in the implanted state at an annulus,

FIG. 5 shows a schematic depiction of a cut along line V-V in FIG. 4 through the aortic wall, with a view of the cusp of the valve implant,

FIG. 6 shows a schematic depiction of the advancement of the implant device with the valve implant according to FIG. 1 to an implantation site,

FIG. 7 shows an alternative valve implant in the implanted state at an annulus,

FIG. 8 shows a further alternative valve implant in the implanted state at an annulus, in a schematic depiction,

FIG. 9 shows a schematic depiction of a mechanical support structure for the valve cusp with the capability of being affixed onto a proximal annular structure,

FIG. 10 shows a third alternative valve implant in the implanted state at an annulus, in a schematic depiction,

FIG. 11 shows a schematic sequence of the crosslinking of unsaturated polyester,

FIG. 12 shows initiator-accelerator combinations in UP resin systems, and

FIG. 13 shows the scheme of peroxide cleavage to start polymerization.

### DETAILED DESCRIPTION

Elements that are functionally identical or similar-acting are labeled using the same reference numerals in the figures. The figures are schematic depictions of the invention. They do not depict specific parameters of the invention. Furthermore, the figures merely show typical embodiments of the invention and are not intended to limit the invention to the embodiments shown.

Regarding elements in a figure that are not described in detail, reference is made to the corresponding description of the elements in preceding figures to avoid unnecessary repetition.

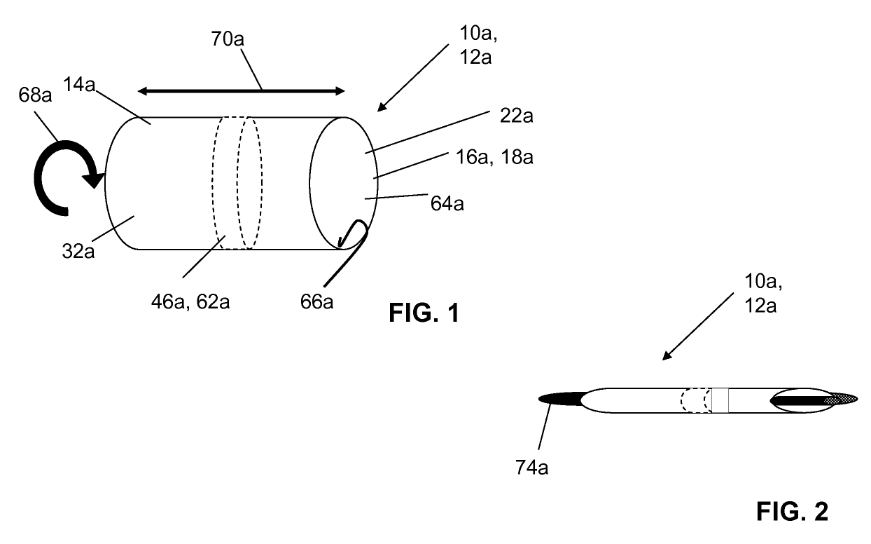

FIG. 1 shows a medical valve implant 10a and a heart valve implant 12a for implantation in an animal body and/or human body in the expanded state. Valve implant 10a comprises a support frame 14a having a wall 16a, wherein support frame 14a is formed by a hollow cylinder 64a in the form of a polymer tube, and wall 16a is formed by the complete cylinder jacket. It would also be feasible in principle for wall 16a to be formed only by an outer wall of hollow cylinder 64a. Support frame 14a comprises a stent 32a or forms a stent 32a, wherein the stent frame thereof is formed entirely by the elastic polymer tube. A valve 46a, designed as aortic valve 62a, is non-detachably fastened to an inner jacket surface 66a of support frame 14a. Wall 16a of support frame 14a comprises a region 18a that extends in a circumferential direction 68a around the entire circumference of support frame 14a and along an entire axial length 70a.

Region 18a and wall 16a are identical in this case. Region 18a and wall 16a are made of a curable material 22a which can be cured using UV radiation 20a. Material 22a is composed of a mixture of highly flexible acrylates and cyanoacrylates, to which triethanolamine, N-vinyl pyrrolidone, and eosin Y have been added. For example, 0.5 ml triethanolamine and 50 μl N-vinyl pyrrolidone in which 0.3% eosin Y is dissolved are added to a quantity of 10 ml acrylates and cyanoacrylates which can be used in any mixing ratio. By using such a composition, the crosslinking reaction can be started with the aid of a UV light source 72a, even without increasing the temperature.

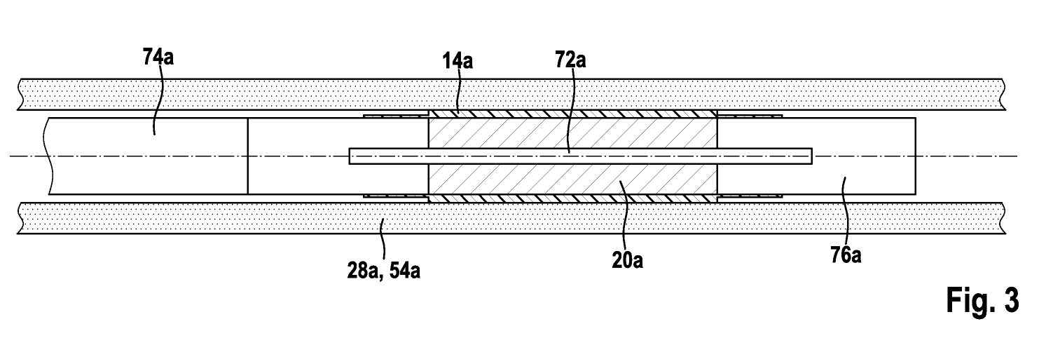

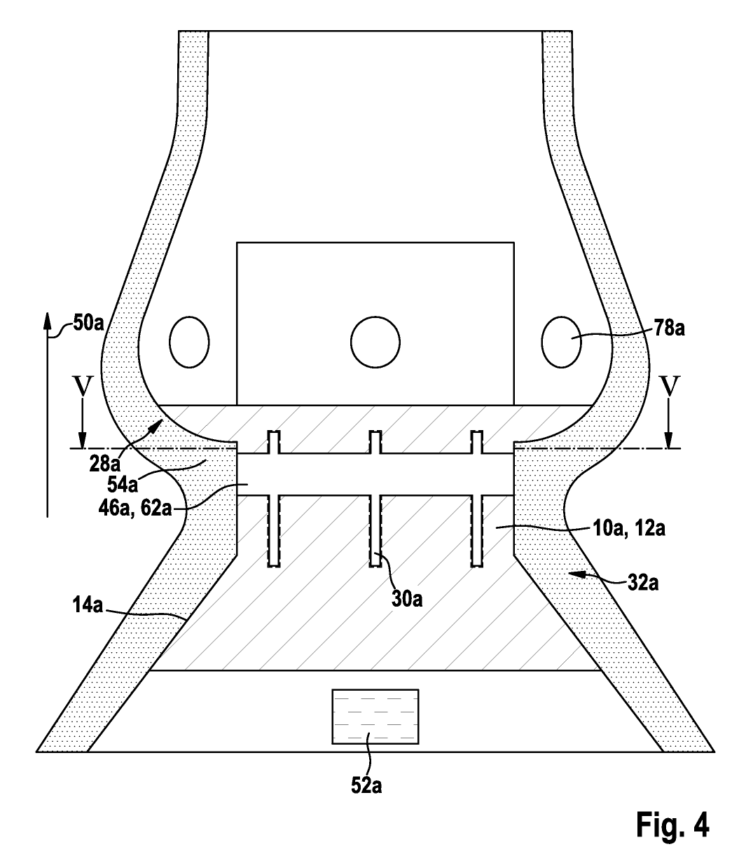

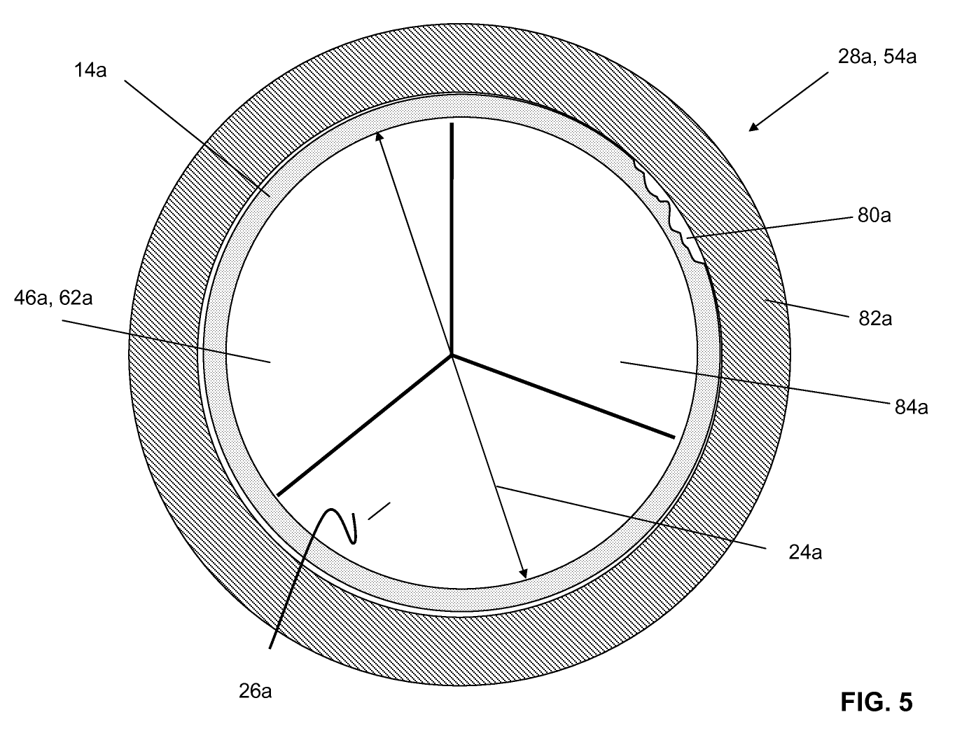

As shown in FIG. 2, valve implant 10a is crimped, in the collapsed state thereof, onto an implantation device or a balloon catheter 74a. FIG. 3 shows a schematic depiction of the implantation device and valve implant 10a during implantation of valve implant 10a at an implantation site 28a, such as an annulus 54a or the aortic annulus of the heart (see FIG. 4). Valve 46a is not shown here, for clarity. During implantation, support frame 14a or the polymer tube is dilated using a balloon 76a at implantation site 28a, and is thereby adapted optimally to a vascular anatomy. A UV light source 72a that is controllable from the outside is positioned in balloon 76a, using which UV radiation 20a is irradiated via balloon 76a with an energy of 1-10 J/cm². As a result, support frame 14a is cured in a time of 1-60 seconds, thereby enabling it to perform its support function. After curing, valve implant 10a—despite the low installation height thereof—is affixed optimally and is connected to annulus 54a in a form-fit manner, which is advantageous in terms of flow mechanics. This is shown in FIG. 4 which shows valve implant 10a implanted at annulus 54a. Due to the short installation height thereof, support frame 14a leaves passages 78a of the coronary arteries open and completely covers the physiological valve (not shown), thereby protecting artificial valve 46a from becoming affected by a calcification 80a on the physiological valve (see FIG. 5).

As an option, as indicated using a dashed line in FIG. 4, support frame 14a can comprise a means 30a for mechanical reinforcement in the form of a metallic structure composed of Nitinol. Means 30a is integrated in the curable polymer. Support frame 14a or the polymer tube extends proximally and distally beyond means 30a. Basically, a plurality of means 30a can be integrated, and they can also be connected to form a framework. It is advantageous e.g. that thorn-type anchoring mechanisms of the type used often for implants according to the prior art are not needed, since they can irritate the tissue and induce inflammation.

As shown in FIG. 5, which is a cut along line V-V in FIG. 4 through an aortic wall 82a with a view of cusp 84a of valve 46a, support frame 14a is provided to compensate for a difference in a shape or a round shape of an inner cross-section 24a of support frame 14a and an e.g. irregular cross-sectional area 26a of implantation site 28a. In the region of a calcification 80a on aortic wall 82a, support frame 14a can adapt to the contour of calcification 80a during implantation, due to flexible and deformable wall 16a thereof, without a round geometry of valve 46a being affected. As a result, cusps 84a can open and close freely.

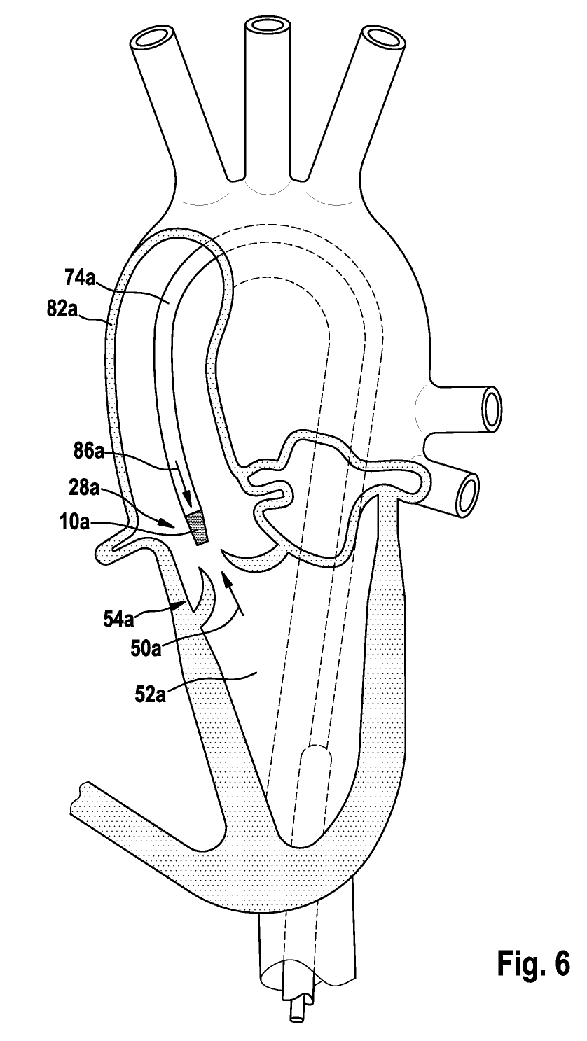

The advancement of medical valve implant 10a using a transfemoral access is illustrated schematically in a partial sectional view, in FIG. 6. The implantation device or balloon catheter 74a with valve implant 10a are advanced to implantation site 28a, e.g. annulus 54a of the natural aortic valve with cusps, in a manner known per se. In this case, an implantation direction 86a is opposite a flow direction 50a of a flow medium 52a such as blood (see FIG. 4). If the position is correct, valve implant 10a is dilated using balloon 76a—as shown in FIG. 3—until it assumes its exact position (see FIG. 4). Once this has taken place, curable material 22a or the polymer is cured using UV radiation 20a, and valve implant 10a is affixed at annulus 54a in the intended end state thereof.

The “transapical” access is also used in clinical practice, in which case the valve is implanted through the ventricle. In that case the terms “proximal” and “distal” would be reversed in the following descriptions.

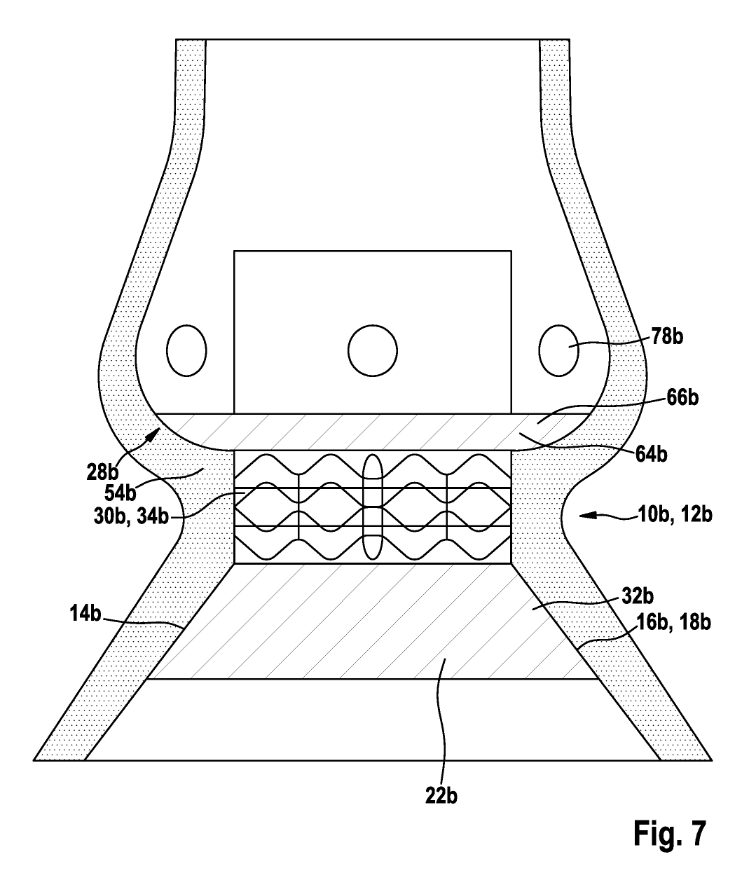

Alternative embodiments of medical valve implant 10a are shown in FIGS. 7 to 10. Components, features, and functions that are essentially the same are labelled using the same reference numerals. To distinguish the exemplary embodiments from each other, the is reference numerals of the exemplary embodiments are appended with the letters a through

d. The description that follows is limited mainly to the differences from the embodiment presented in FIGS. 1 to 6, and reference is made to the description of the embodiment shown in FIGS. 1 to 6 with regard for the components, features, and functions that remain the same.

FIG. 7 shows, in a schematic view, an alternative medical valve implant 10b or a heart valve implant 12b for implantation in an animal and/or human body with an artificial valve (not depicted) in the implanted state at an implantation site 28b or an annulus 54b of a natural aortic valve. Valve implant 10b comprises a support frame 14b having a wall 16b, region 18b of which—which corresponds to wall 16b and entire support frame 14b—comprises a material 22b that can be cured using UV radiation.

In addition to stent 32b, which is formed by support frame 14b itself, valve implant 10b or support frame 14b comprises a further stent 34b as a means 30b for mechanical reinforcement. Stent 34b is disposed on an inner jacket surface 66b of hollow cylinder 64b of support frame 14b and is likewise balloon-dilatable. Basically, stent 34b could also be designed to be self-expandable. In addition, the artificial valve is fastened to stent 32b.

Support frame 14b or the polymer tube extends proximally and distally beyond stent 34b, but not to passages 78b of the coronary arteries, and therefore the polymer tube is adapted proximally and distally of stent 34b to the individual morphology when dilated. Once it is positioned exactly, it is cured using a UV light source.

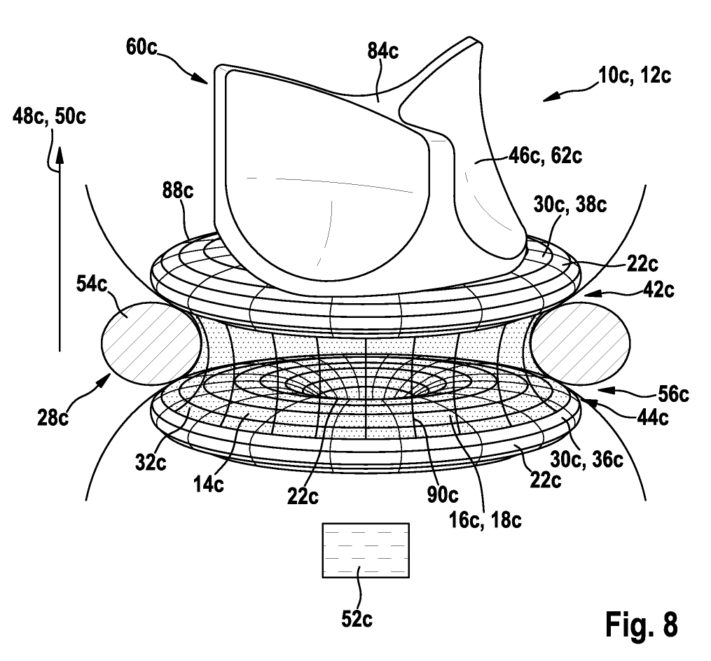

FIG. 8 shows a further alternative valve implant 10c or a heart valve implant 12c for implantation in an animal body and/or human body with an artificial valve 46c in the form of an aortic valve 62c and a support frame 14c. Support frame 14c, which is formed by an elastic polymer tube, comprises a wall 16c, region 18c of which—which corresponds to wall 16c and entire support frame 14c—is made of a material 22c that can be cured using UV radiation. Support frame 14c, which forms a stent 32c, comprises a means 30c on proximal end 42c and on distal end 44c for mechanical reinforcement, each means 30c being formed by an annular structure 36c, 38c. In the intended end state i.e. in the implanted state at an implantation site 28c, the two annular structures 36c, 38c are disposed axially upstream of and downstream of an annulus 54c in flow direction 50c of a flow medium 52c. Furthermore, each annular structure 36c, 38c is formed by a hollow ring, jacket 88c of which is composed of a flexible polymer such as polyethylene.

Annular structures 36c, 38c can be filled with a curable material 22c (materials, see below), and can be filled independently of each other. Annular structure 36c on distal end 44c is filled first using the catheter system connected to valve implant 10c. It can be gradually positioned in an optimal manner by intentionally inflating it slowly. Once distal annular structure 36c is seated correctly, annular structure 38c on proximal end 42c is positioned and filled in an analogous manner. In the case of both procedures, the solution used for filling can be partially withdrawn using the catheter system, to lower the pressure if necessary. This makes it possible to easily reposition the implant if it should become mispositioned e.g. loss of seal integrity. Annular structures 36c, 38c are therefore positioning means. In general, annular structures 36c, 38c can be filled with the same material 22c or different materials 22c.

A communication structure 56c in the form of a channel system having hollow elements 90c is disposed between the two annular structures 36c, 38c, thereby enabling material 22c to be distributed between the rings. Hollow elements 90c can be integrated in support frame 14c or formed by a separate structure (not depicted). As an alternative to the channel system, support frame 14c can also have a double-wall design, and so a cavity is formed between the walls, via which annular structures 36c, 38c can communicate. Openings must be formed in jackets 88c of the rings for material to be exchanged (not depicted). As an alternative, annular structures 36c, 38c can also be integrated into a hollow, fillable polymer foil as bulges on ends 42c, 44c. Furthermore, communication structure 56c can also be omitted entirely.

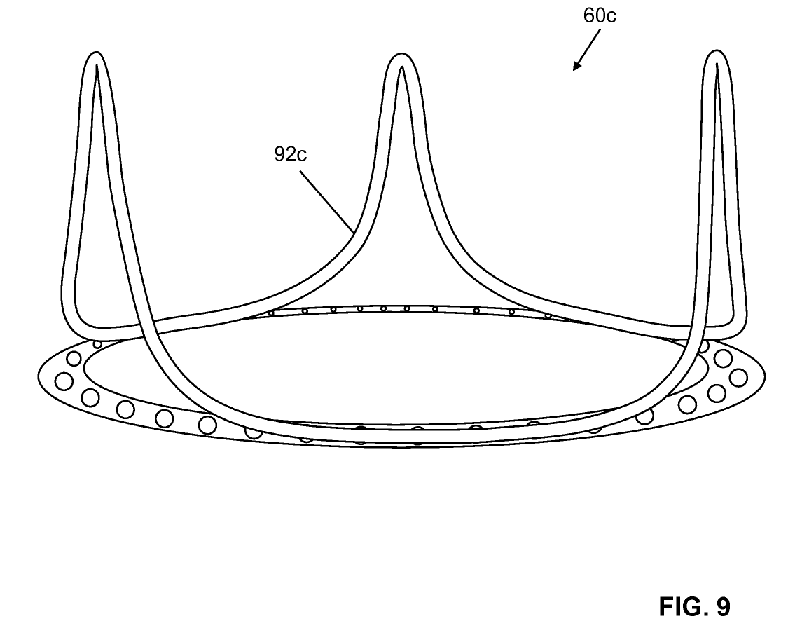

Valve 46c is disposed above annular structure 38c in an axial direction 48c on proximal end 42c of support frame 14c in the aorta above annulus 54c. For upper fixation of cusps 84c of valve 46c, a mechanical support structure 60c in the form of a metal framework 92c, made e.g. of Nitinol, is fastened to proximal annular structure 38c (see FIG. 9). This ensures the relative positioning of annular structure 38c and cusp 84c and ensures that cusp 84c can open and close reliably.

Curable material 22c for filling annular structures 36c, 38c can be cured using UV radiation or it can harden itself after a certain period of time. A plurality of alternative polymer solutions are feasible for this purpose:

### Example 1

### UV-Curable Polymer Solution

Granulated polymethacrylate (PMMA; 1-10 μm) is mixed with methylmethacrylate (composed of 97.6% methylmethacrylate (MMA), 2% N,N-dimethyl-p-toluidine and 20 ppm hydroquinone) and polyisobutylene (PIB; M_{w}, 300-3000 g/mol). The following are used: 20% PMMA, 50% PIB and 30% MMA. The PIB acts a molecular softening agent, and PMMA acts as filler which makes it possible to use smaller quantities of MMA. Pure MMA would polymerize and create a considerable amount of heat (Trommsdorff effect). 25 ml triethanolamine and 5-10 ml N-vinyl pyrrolidone in which 0.3% eosin Y was dissolved are added to this mixture.

### Example 2

### Self-Curing Polymer Solution

A liquid phase is composed of 97.6% methylmethacrylate (MMA) as monomer, 2.4% N,N-dimethyl-p-toluidine as activator, and 20 ppm hydroquinone as stabilizer. The polymer powder is composed of 64.4% poly(methylmethacrylate) (PMMA), 0.6% dibenzoyl peroxide as the initiator of polymerization, 25% barium sulfate as x-ray marker, and 10% filler. Mechanically inelastic material such as hydroxylapatite in bone cement can be used as the filler. As a result the cured annular structures are mechanically highly rigid and similar to bone. Furthermore, a mechanically elastic material such as rubber particles can be used as filler. The proportion thereof can be used to obtain specific mechanical properties of the cured annular structure.

As prepolymers, polyisobutylene as well as butyl rubber (isobutene-isoprene rubber; IIR) are suitable, in particular IIR obtained via cationic polymerization from precipitation polymerization. Further prepolymers or lattices can be created easily using suspension polymerization. The particle size can be between 2-200 μm. Particles in the range 10-50 μm are preferable. A good overview of plastisol or organosols is provided in WO 2008/019899. In that publication, a few methacrylate compounds are described that harden after processing. After the liquid phase is combined with the other substances, polymerization is initiated and the material cures within approximately 10 minutes.

### Example 3

### Self-Curing Polymer Solution

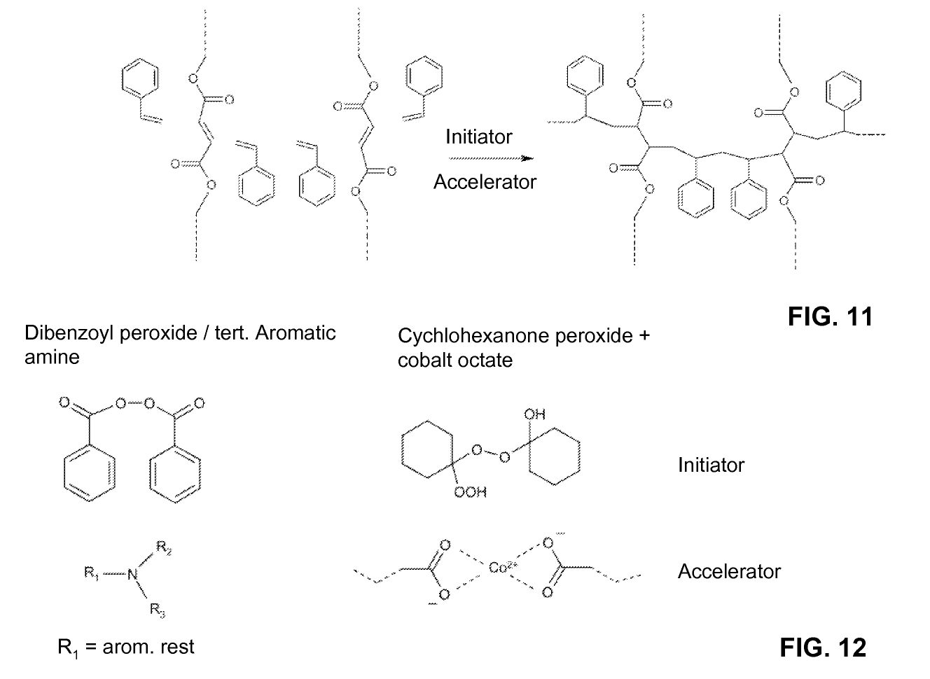

Another example of a self-curing polymer is unsaturated polyester (UP). The mode of action is illustrated in FIG. 11. The UP contains monomers (styrene in the present example). Crosslinking and stiffening take place via a subsequent, UV-induced crosslinking reaction, in which case intra- or intermolecular crosslinkings of the UP are feasible. Although the styrene or the other monomers used also function as diluting agents for adjusting the processing viscosity, they should not be considered to be solvents in the actual sense since they become a component of the polymeric network by the chemical hardening reaction. They are therefore better known as “reactive diluters”. The loss due to evaporation is typically very low. The manufacture of UPs is known. They are formed using condensation reactions of unsaturated dicarbonic acids or unsaturated carbonic acid anhydrides with dioles. The preferred molar mass is 6,500-13,000 g/mol. When 112 ml of the UP (10,000 g/mol) is used, 16.5 ml styrene are added. Hardening at body temperature normally requires the addition of an “accelerator” to the unsaturated polyester system since hardening would not occur otherwise, or the curing time could take days to weeks. Two main initiator-accelerator combinations exist to accelerate UP resin systems, namely conventional hardening of UP resins using dibenzoyl peroxide/tert. aromatic amine, and using cyclohexanone/cobalt octoate, as shown in FIG. 12.

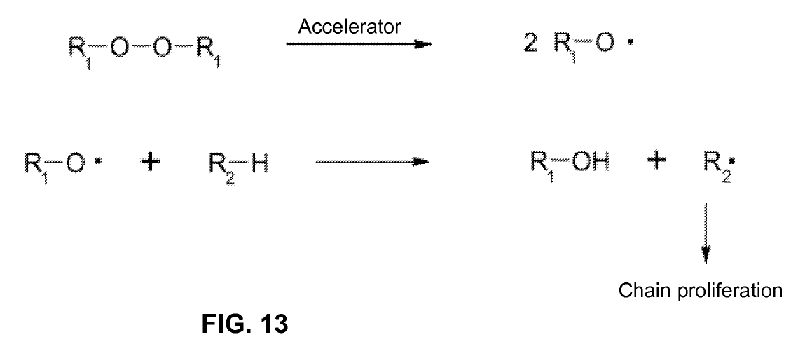

Initiators are understood in macromolecular chemistry to be organic peroxides which can initiate the polymerization of unsaturated compounds by their decomposition into radicals. At the typical processing temperatures of conventionally hardened, unsaturated polyester formulations, radical formation takes place not at all or very slowly without accelerators. The “accelerators” catalyze the decomposition of peroxides by reducing the dissociation energy and initiate the reaction shown in FIG. 13.

The conventional hardening of UP resins using dibenzoyl peroxide/tert. aromatic amine takes place as follows: The decomposition of diacyl peroxides such as dibenzoyl peroxide is catalyzed by tertiary aromatic amines. This system has high reactivity and is therefore suitable for hardening unsaturated polyester formulations even at relatively low temperatures (to approximately 5° C.). The peroxide hardener can be added subsequently to the polymer tube as the final component; this ensures that the UP is present in non-reactive form. Pure peroxide would be too dangerous to handle because it easily decomposes at temperatures above 25° C. or if acted upon by direct sunlight. The appropriate quantity of hardener (0.015 mol for the formulation indicated above) in a solvent (ethanol; 2-3%) is therefore added to the tube structure shortly before processing; the natural contraction reactions of the cardiac muscle are sufficient for mixing. The processing time of such a system is normally set to approximately 4-6 minutes.

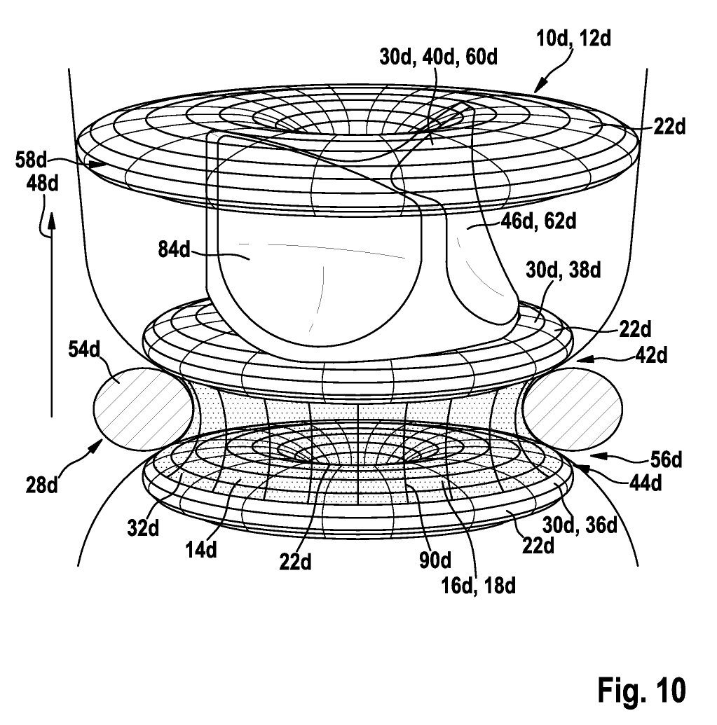

FIG. 10 shows a third alternative valve implant 10d and a heart valve implant 12d for implantation in an animal body and/or human body with an artificial valve 46d or aortic valve 62d and a support frame 14d, which is designed as a stent 32d. Support frame 14d, which is formed by an elastic polymer tube, comprises a region 18d that is made of a material 22d that can be cured using UV radiation, wherein region 18d corresponds to a wall 16d and entire support frame 14d. Valve 46d is disposed above an annular structure 38d in an axial direction 48d on a proximal end 42d of support frame 14d in the aorta over an implantation site 28d or an annulus 54d. For upper fixation of cusps 84d of valve 46d a mechanical support structure 60d in the form of a third annular structure 40d is disposed on proximal end 58d of valve 46d. Annular structure 40d has the same design as means 30d for reinforcement or annular structures 36d, 38d on proximal end 42d and on distal end 44d, and can be filled with the same curable material 22d as annular structures 36d, 38d or with another material 22d. Furthermore, a communication structure having hollow elements 90d between annular structures 36d, 38d can be provided between annular structure 40d and one or both annular structures 36d, 38d (not depicted).

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE CHARACTERS

10 Valve implant
12 Heart valve implant
14 Support frame
16 Wall
18 Region
20 UV radiation
22 Material
24 Inner cross-section
26 Cross-sectional area
28 Implantation site
30 Means
32 Stent
34 Stent
36 Annular structure
38 Annular structure
40 Annular structure
42 End
44 End
46 Valve
48 Direction
50 Flow direction
52 Flow medium
54 Annulus
56 Communication structure
58 End
60 Support structure
62 Aortic valve
64 Hollow cylinder
66 Jacket surface
68 Circumferential direction
70 Length
72 Light source
74 Balloon catheter
76 Balloon
78 Passage
80 Calcification
82 Aortic wall
84 Cusp
86 Implantation direction
88 Jacket
90 Element
92 Metal framework

What is claimed is:

1. A medical valve implant, in particular a heart valve implant, for implantation in an animal body or human body, having a support frame which has at least one wall, characterized in that at least one region of the at least one wall of the support frame is curable.

2. The medical valve implant according to claim 1, characterized in that the region of the at least one wall of the support frame is curable using UV radiation.

3. The medical valve implant according to claim 1, characterized in that the at least one region of the at least one wall of the support frame is made of a curable material selected from a group consisting of an acrylate, a methacrylate, a cyanoacrylate, an epoxide, a urethane, an acrylamide, and an acyl acid.

4. The medical valve implant according to claim 1, characterized in that the support frame is provided to compensate for a difference in a shape of an inner cross section of the support frame and a cross-sectional area of an implantation site.

5. The medical valve implant according to claim 1, characterized in that the support frame comprises a stent.

6. The medical valve implant according to claim 1, characterized in that the support frame comprises at least one means for mechanical reinforcement.

7. The medical valve implant according to claim 6, characterized in that the means for mechanical reinforcement is formed by at least one annular structure.

8. The medical valve implant according to claim 7, characterized in that the annular structure is disposed on a proximal end or a distal end, optionally both, of the support frame.

11. The medical valve implant according to claim 8, characterized by a valve which is disposed above the annular structure in an axial direction on the proximal end of the support frame.

9. The medical valve implant according to claim 7, characterized in that the annular structure can be filled with a curable material.

10. The medical valve implant according to claim 1, characterized by at least two annular structures which can be filled independently of each other.

12. The medical valve implant according to claim 1, characterized in that at least two annular structures are disposed axially upstream of and downstream of an annulus in the flow direction of a flow medium in the intended end state.

13. The medical valve implant according to claim 1, characterized in that at least one communication structure is disposed between two annular structures.

14. The medical valve implant according to claim 1, characterized in that a support structure is disposed at least on an annular structure on a proximal end of the support frame or on a proximal end, optionally both, of a valve.

15. The medical valve implant according to claim 1, characterized by being an aortic valve.
